(19) United States
(12) Patent Application Publication
(10) Pub. No.: US 2011/0160726 A1
(43) Pub. Date: Jun. 30, 2011

(54) APPARATUS AND METHODS FOR FLUID COOLED ELECTROPHYSIOLOGY PROCEDURES

(76) Inventor: Frank Ingle, Palo Alto, CA (US)

(21) Appl. No.: 12/916,500

(22) Filed: Oct. 30, 2010

Related U.S. Application Data
(60) Provisional application No. 61/291,134, filed on Dec. 30, 2009.

(51) Int. Cl. A61B 18/14

(52) U.S. Cl. 606/49; 606/41

(57) ABSTRACT

Methods and apparatus associated with irrigated tissue ablation procedures.

### CROSS-REFERENCE TO RELATED APPLICATIONS

This application claims the benefit of U.S. Provisional Application Ser. No. 61/291,134, filed Dec. 30, 2009 and entitled “Apparatus and Methods for Fluid Cooled Electrophysiology Procedures,” which is incorporated herein by reference.

### BACKGROUND

1. Field

The present apparatus and methods relate generally to the formation of lesions in tissue.

2. Description of the Related Art

There are many instances where electrodes are inserted into the body. One instance involves the treatment of cardiac conditions such as atrial fibrillation, atrial flutter and ventricular tachycardia, which lead to an unpleasant, irregular heart beat, called arrhythmia. Atrial fibrillation, flutter and ventricular tachycardia occur when anatomical obstacles in the heart disrupt the normally uniform propagation of electrical impulses in the atria. These anatomical obstacles (called “conduction blocks”) can cause the electrical impulse to degenerate into several circular wavelets that circulate about the obstacles. These wavelets, called “reentry circuits,” disrupt the normally uniform activation of the chambers within the heart.

A variety of minimally invasive electrophysiological procedures employing catheters that carry one or more electrodes have been developed to treat conditions within the body by ablating soft tissue (i.e. tissue other than blood and bone). Soft tissue is simply referred to as “tissue” herein and references to “tissue” are not references to blood. With respect to the heart, minimally invasive electrophysiological procedures have been developed to treat atrial fibrillation, atrial flutter and ventricular tachycardia by forming therapeutic lesions in heart tissue. The formation of lesions by the coagulation of soft tissue (also referred to as “ablation”) during minimally invasive surgical procedures can provide the same therapeutic benefits provided by certain invasive, open-heart surgical procedures. In particular, the lesions may be placed so as to interrupt the conduction routes of reentry circuits.

The catheters employed in electrophysiological procedures typically include a relatively long and relatively flexible shaft that carries a distal tip electrode and, in some instances, one or more additional electrodes near the distal end of the catheter. The proximal end of the catheter shaft is connected to a handle which may or may not include steering controls for manipulating the distal portion of the catheter shaft. The length and flexibility of the catheter shaft allow the catheter to be inserted into a main vein or artery (typically the femoral artery), directed into the interior of the heart where the electrodes contact the tissue that is to be ablated. Fluoroscopic imaging may be used to provide the physician with a visual indication of the location of the catheter. Exemplary catheters are disclosed in U.S. Pat. Nos. 6,013,052, 6,203,525, 6,214,002 and 6,241,754.

The tissue coagulation energy is typically supplied and controlled by an electrosurgical unit (“ESU”) during the therapeutic procedure. More specifically, after an electrophysiology device has been connected to the ESU, and one or more electrodes or other energy transmission elements on the device have been positioned adjacent to the target tissue, energy from the ESU is transmitted through the electrodes to the tissue to from a lesion. The amount of power required to coagulate tissue ranges from 5 to 150 W. The energy may be returned by an electrode carried by the therapeutic device, or by an indifferent electrode such as a patch electrode that is secured to the patient's skin.

Tissue charring due to overheating, thrombus and coagulum formation, and tissue popping, which occurs when subsurface temperature levels exceed 100° C. and tissue vaporizes, are sometimes associated with soft tissue coagulation. In order to, among other things, prevent tissue charring and thrombus/coagulum formation, a variety of electrophysiology systems employ fluid to cool the electrode (or electrodes) and/or the tissue adjacent to the electrodes. In some systems, which are referred to as “open irrigation systems,” fluid exits the electrophysiology device through outlets in the catheter shaft and/or outlets in the electrode. The fluid cools the electrode and adjacent tissue to prevent charring and tissue vaporization, prevents thrombus formation by diluting the blood that comes into contact with the electrode, and also prevents coagulation on the electrode. In some systems, fluid is supplied to the catheter at a constant rate (e.g. 20-30 ml/min.) during tissue coagulation, while in others the rate is varied in an attempt to maintain a preset tissue temperature. The fluid may also be conductive in some instances and, accordingly, the fluid also provides an electrical path for coagulation energy. “Closed irrigation systems” are similar in that fluid is used to cool the electrode. Here, however, the fluid does not exit the catheter and is instead returned to the proximal region of the catheter and vented therefrom.

The present inventor has determined that conventional irrigated electrophysiology systems are susceptible to improvement. For example, clinicians frequently estimate lesion depth based on the level of power supplied to the electrode by the power supply and the length of time that the power is supplied. The power supply is set to a power level and power duration that corresponds to the desired lesion depth prior to the ablation procedure. While this may be appropriate in the context of non-irrigated catheters that are configured such that the electrode is not substantially exposed to the blood pool and essentially all of the energy supplied to the electrode is dissipated into the tissue, the present inventor has determined that it is less appropriate in the context of irrigated systems. Specifically, some of the energy delivered to the electrode by the power supply in irrigated systems is lost to irrigation fluid instead of being dissipated into the tissue. The present inventor has also determined that it is difficult to accurately quantify the magnitude of the energy loss and, by extension, the level of energy actually dissipated into the tissue, using conventional systems. The inability to accurately quantify level of energy actually dissipated into the tissue can result in under-delivery of energy to the tissue (and lesions of insufficient depth) and over-delivery of energy to the tissue (and tissue charring and pops).

### SUMMARY

Methods and apparatus in accordance with at least some of the present inventions involve transferring substantially all of the heat flowing from the tissue to the tip electrode to the irrigation fluid. The associated increase in the temperature of the irrigation fluid may be used to determine the amount of energy lost to the irrigation fluid and, by extension, the amount of power actually supplied to (or “dissipated in”) the tissue. Such methods and apparatus provide a number of advantages over conventional methods and apparatus. For example, the present methods and apparatus allow the clinician and/or the power supply and/or the fluid supply to accurately quantify level of energy actually dissipated into the tissue, adjust power or fluid flow rates accordingly, and reduce the likelihood of under-delivery or over-delivery of energy to the tissue.

The above described and many other features and attendant advantages of the present inventions will become apparent as the inventions become better understood by reference to the following detailed description when considered in conjunction with the accompanying drawings.

### BRIEF DESCRIPTION OF THE DRAWINGS

Detailed description of exemplary embodiments will be made with reference to the accompanying drawings.

FIG. 1 is a perspective view of an electrophysiology system in accordance with one embodiment of a present invention.

FIG. 2 is a partial section view showing a lesion being formed by the electrophysiology system illustrated in FIG. 1.

FIG. 3 is a section view take along line 3-3 in FIG. 1.

FIG. 4 is a section view take along line 4-4 in FIG. 1.

FIG. 5 is an elevation view of an electrophysiology electrode in accordance with one embodiment of a present invention.

FIG. 6 is a section view take along line 6-6 in FIG. 5.

FIG. 7 is a section view take along line 7-7 in FIG. 5.

FIG. 8 is an elevation view of an electrophysiology electrode in accordance with one embodiment of a present invention.

FIG. 9 is a section view take along line 9-9 in FIG. 8.

FIG. 10 is a section view take along line 10-10 in FIG. 8.

FIG. 11 is a section view take along line 11-11 in FIG. 8.

FIG. 12 is an end view of a portion of the device illustrated in FIG. 8.

FIG. 13 is a partial section view showing a lesion being formed by device illustrated in FIGS. 8-12.

### DETAILED DESCRIPTION OF EXEMPLARY EMBODIMENTS

The following is a detailed description of the best presently known modes of carrying out the inventions. This description is not to be taken in a limiting sense, but is made merely for the purpose of illustrating the general principles of the inventions.

The present inventions have application in the treatment of conditions within the heart, gastrointestinal tract, prostrate, brain, gall bladder, uterus, and other regions of the body. With regard to the treatment of conditions within the heart, the present inventions may be associated with the creation of lesions to treat atrial fibrillation, atrial flutter and ventricular tachycardia.

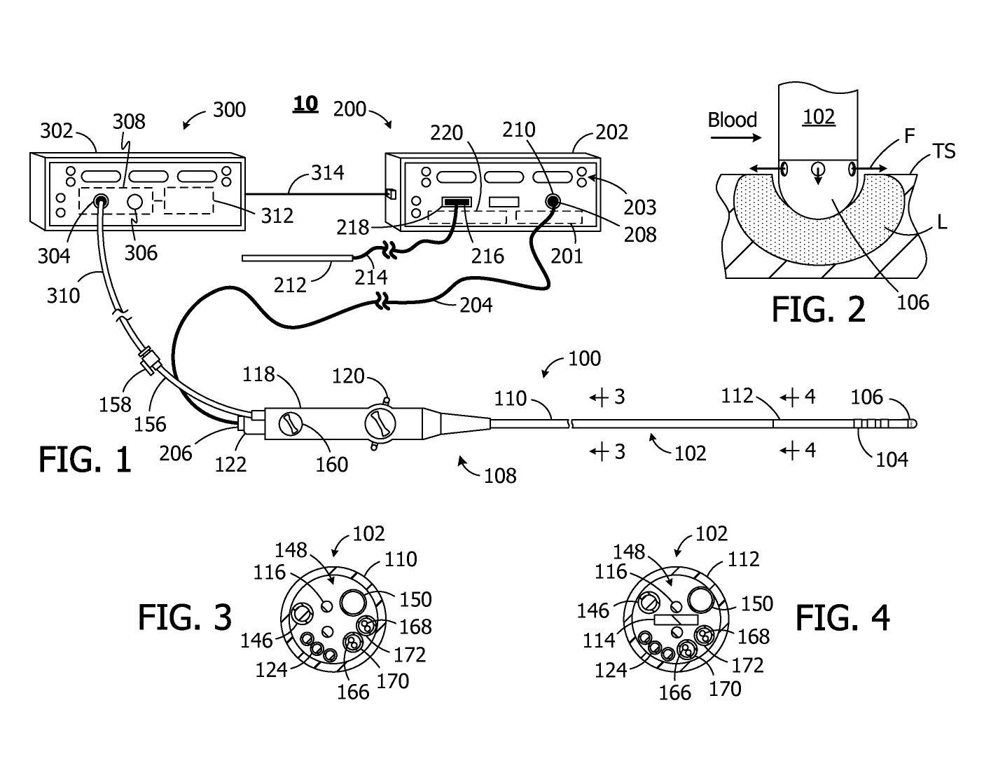

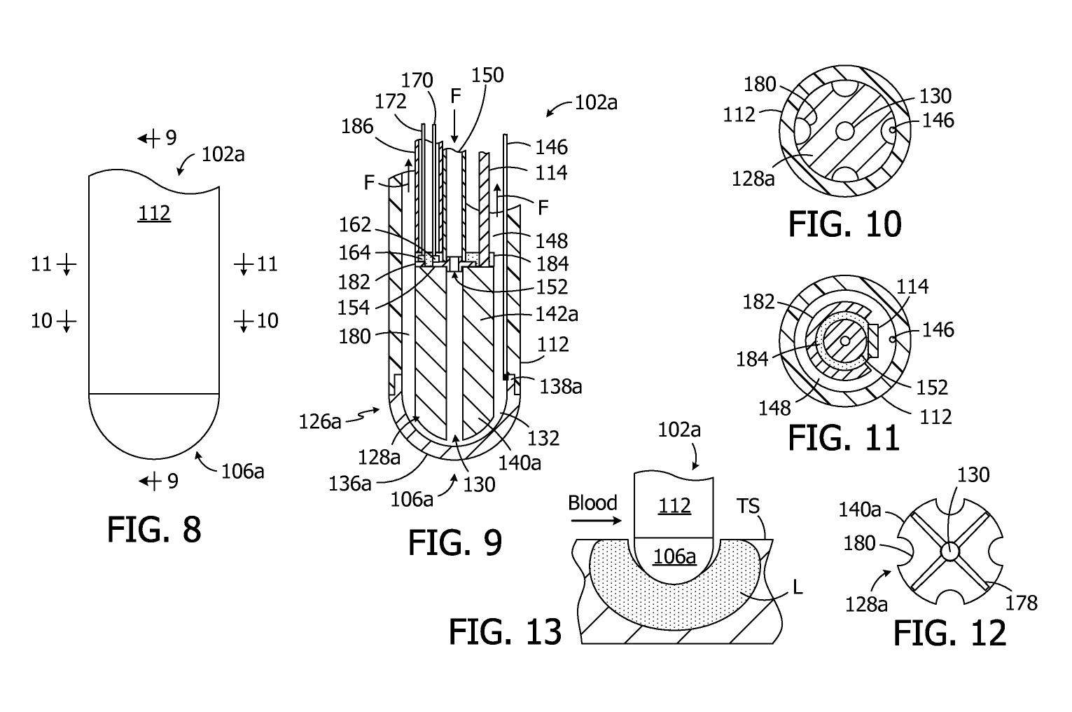

A tissue coagulation system 10 in accordance with one embodiment of a present invention is illustrated in FIG. 1. The exemplary system 10 includes a catheter apparatus 100, a power supply and control apparatus 200 that supplies RF current to the ablation electrode(s) based on power, temperature and time settings as well as the temperature, RF power and impedance, and a fluid supply and control apparatus 300 that supplies cooling fluid to the catheter apparatus during coagulation procedures at constant flow rates selected by the clinician or at flow rates that vary based on feedback from the ablation electrode. The tissue coagulation system 10 may be used to perform an open irrigation tissue coagulation procedure, where fluid F exits a tip electrode 106 on the catheter apparatus 100 in the manner illustrated for example in FIG. 2, to create a lesion L in a tissue surface TS. Where other catheter apparatus are employed, and as is discussed below with reference to FIGS. 8-13, closed irrigation tissue coagulation procedures may be performed.

The tip electrode 106 (and 106a in FIGS. 8-13) is configured such that substantially all of the heat flowing from the tissue to the tip electrode is transferred to the irrigation fluid. As a result, the measured increase in the temperature of the irrigation fluid as it passes through the tip electrode may be used by the power supply and control apparatus 200 in the manner described below to determine the amount of energy flowing from the tissue to the fluid passing through the tip electrode (P_{LOST}). The power actually supplied to (or “dissipated in”) the tissue (P_{TISSUE}) is equal to the power supplied by the power supply and control apparatus 200 to the tip electrode (P_{SUPPLIED}) less the power lost to the tip electrode (P_{LOST}). The calculated power that is actually dissipated is tissue (P_{TISSUE}) facilitates more accurate estimations of the temperature distribution within the tissue and, therefore, lesion depth, than can be realized with conventional systems that merely measure the temperature of the tissue surface adjacent to the tip electrode.

It should be noted that the system illustrated in FIGS. 1 and 2 is merely one example of a tissue coagulation system with which the present inventions may be associated. The present inventions are applicable to any and all open and closed irrigated coagulation systems, including those yet to be developed and those that are not catheter based, as well as to the individual components thereof.

The exemplary catheter apparatus 100 illustrated in FIG. 1 includes a hollow, flexible catheter 102, a plurality of ring electrodes 104, a tip electrode 106, and a handle 108. The catheter 102 may be steerable and formed from two tubular parts, or members, both of which are electrically non-conductive. The proximal member 110 is relatively long and is attached to the handle 108, while the distal member 112, which is relatively short, carries the electrodes 104 and 106. The exemplary catheter 102 is also configured for use within the heart and, accordingly, is about 6 French to about 10 French in diameter and the portion that is inserted into the patient is typically about 60 to 160 cm in length. The exemplary catheter apparatus 100 is steerable and, to that end, is provided with a conventional steering center support and steering wire arrangement. Referring to FIGS. 1, 3 and 4, the proximal end of the exemplary steering center support 114 is mounted near the distal end of the proximal member 110, while the distal end of the steering center support is secured to the tip assembly 126 (FIG. 6) in the manner described below. A pair of steering wires 116 are secured to opposite sides of the steering center support 114 and extend through the catheter body 102 to the handle 108, which is also configured for steering. More specifically, the exemplary handle 108 includes a handle body 118 and a lever 120 that is rotatable relative to the handle body. The proximal end of the catheter 102 is secured to the handle body 118, while the proximal ends of the steering wires 116 are secured to the lever 120. Rotation of the lever 120 will cause the catheter distal member 112 to deflect relative to the proximal member 110.

The exemplary ring electrodes 104, which may be used for electrical sensing or tissue coagulation, are connected to an electrical connector 122 on the handle 108 by signal wires 124. Electrically conducting materials, such as silver, platinum, gold, stainless steel, plated brass, platinum iridium and combinations thereof, may be used to form the electrodes 104. The diameter of the exemplary electrodes 104 will typically range from about 5 French to about 11 French, while the length is typically about 1 mm to about 4 mm with a spacing of about 1 mm to about 10 mm between adjacent electrodes.

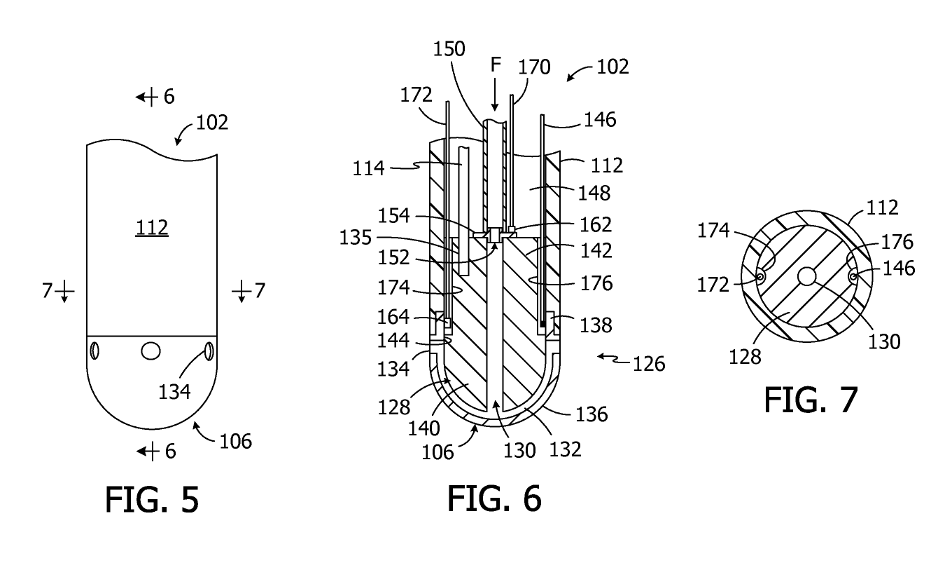

Turning to FIGS. 5-7, the exemplary catheter apparatus 100 is provided with a tip assembly 126 (or “electrode assembly”) that includes the aforementioned tip electrode 106 and an insulation member 128 that provides thermal and electrical insulation. The elements of the tip assembly 126 individually and/or together perform a variety of functions including, but not limited to, transmitting coagulation energy to tissue, providing a fluid flow path that allows the fluid to be heated prior to exiting the tip assembly, measuring the increase in temperature of the fluid that passes through the tip assembly, and measuring tissue temperature. To that end, and as discussed in greater detail below, the exemplary tip assembly 126 includes an inlet lumen 130, a fluid heating space 132 connected to the inlet lumen, and a plurality of fluid outlets 134 connected to the fluid heating space. The inlet lumen 130, which extends through the insulation member 128, is connected to the outlets 134 by the fluid heating space 132. The fluid heating space 132 is defined by a space between the tip electrode 106 and the insulation member 128. The insulation member also includes a slot 135 in which the steering center support 114 is mounted.

In the illustrated embodiment, the tip electrode 106 includes a tissue contact portion 136 and a base portion 138. The tissue contact portion 136 is relatively thin to promote heat transfer from the tissue to the irrigation fluid within the heating space 132. The tissue contact portion 136 is also hemispherical-shaped in the illustrated embodiment although other shapes, such as a relatively flat distal end with a rounded edge, may be employed. The fluid outlets 134 are formed in the base portion 138, which is also used to mount the tip electrode to the catheter 102. In the illustrated embodiment, the base portion 138 is relatively short so that only a small portion of the tip electrode 106 will be exposed to blood, and the convective cooling effects thereof, during ablation procedures (FIG. 2). In other embodiments where the tip electrode is longer, or where electrode assemblies proximal of the tip are used to coagulate tissue, thermal insulation may be provided on the portion of the electrodes that will be exposed to blood.

The exemplary insulation member 128 includes a hemispherical portion 140 and a cylindrical portion 142, and the inlet lumen 130 extends though both portions. The hemispherical portion 140 is slightly smaller in diameter than the electrode tissue contact portion 136 so, when the two are positioned relative to one another in the manner illustrated in FIG. 6, the fluid heating space 132 is defined therebetween. The outer diameter of the insulation member cylindrical portion 142 is substantially equal to the inner diameters of the catheter distal member 112 and the tip electrode base portion 138. As such, the insulation member cylindrical portion 142 may be mounted within the catheter distal member 112 (as shown) and a seal 144 is formed between the insulation member cylindrical portion and the inner surface of the tip electrode base portion 138. The tip assembly electrode 106 and insulation member 128 may be secured to the catheter distal member 112 through the use of adhesive or other suitable instrumentalities.

The configuration of the tip assembly (or “electrode assembly”) 126 is such that essentially all of the heat which is transferred from the tissue into the tip electrode 106 is transferred to the irrigation fluid as it passes through the fluid heating space 132. More specifically, the irrigation fluid is heated by convection within the fluid heating space 132 and essentially all of the heat from the tissue to the tip is transferred to the fluid. For example, the fluid heating space 132 within the tip electrode 103 is relatively thin and of low volume as compared to overall volume defined by the outer surface of the electrode. This configuration allows the inlet and outlet temperature of the fluid to be used to calculate the amount of energy flowing from the tissue to the tip electrode 106 (and irrigation fluid) as is described below.

Also, in some instances, the temperature of the fluid when it enters the tip electrode 106 will be about equal to body temperature (i.e. about 37° C.) and the clinician will regulate the irrigation fluid flow rate such that the fluid temperature at the outlets 134 will be about 5° C. higher than the inlet temperature (i.e. about 42° C.). The tissue contact portion 136 is thin and of relatively high thermal conductivity and, accordingly, there is no temperature difference across the tissue contact portion. The temperature of the tissue surface is equal to the temperature of the tissue contact portion 136 that it is in contact with. Thus, in the present example, the tissue temperature is about 37° C. at the center of the tissue contact portion 136 and is about 42° C. at the base portion 138. Those two temperatures and the calculated magnitude of the power being dissipated into the tissue allows a three-dimensional temperature versus depth profile to be calculated. This information may be displayed (e.g. a three-dimensional temperature versus depth profile on a screen), or otherwise communicated, so that the clinician will be able to identify the lesion depth by identifying the depth at which tissue is 50° C. or higher. It should also be noted that although the surface temperature of the tissue is below 50° C. during the procedure (i.e. application of power and irrigation fluid), the surface tissue will be heated to temperatures above 50° C. by the hotter sub-surface tissue when the procedure ends.

With respect to materials and dimensions, the exemplary tip electrode 106 may be formed from any suitable electrically conductive material. By way of example, but not limitation, suitable materials for the main portion of the tip electrode 106 include silver, platinum, gold, stainless steel, plated brass, platinum iridium and combinations thereof. The exemplary tip electrode 106, which is generally hemispherical in shape may, in some exemplary implementations sized for use within the heart, be from about 3 French to about 11 French (about 1 mm to about 4 mm) in diameter and about 3 mm to about 8 mm in length. The fluid outlets 134 are generally circular in shape and are about 0.25 mm to 1 mm in diameter. Although the number of fluid outlets 134 will depend on the intended application (e.g. from 3 to 8), there are six fluid outlets in the illustrated embodiment. The wall thickness of the electrode tissue contact portion 136 may be about 0.1 mm to about 0.5 mm, and the distance between the outer surface of insulation member hemispherical portion 140 and the inner surface of the electrode tissue contact portion (i.e. the thickness of the relatively thin fluid heating space 132) may be about 0.05 mm to about 0.2 mm. The insulation member 128 may be about 5 mm to 10 mm in length, about 0.5 mm to 3 mm in diameter, and formed from electrically and thermally insulating material such as polycarbonate or other plastics commonly used in catheter apparatus. The diameter of the inlet lumen 130 is about 0.25 to 1 mm.

In some instances, the tip assembly 126 may be modified as necessary or desired to insure that all of the heat from the tissue is transferred to the fluid. By way of example, but not limitation, a raised or indented spiral pattern may be formed on the inner surface of the electrode 106 and/or the outer surface of the insulation member hemispherical portion 140 in order to increase the heat transfer effectiveness within the fluid heating space 132. Also, it should be noted that although the present fluid heating space 132 is generally hemispherical, the configuration of the tip electrode and/or insulation member 128 may be adjusted to adjust the shape of the fluid heating space. By way of example, but not limitation, a flat fluid heating space may be employed in some embodiments.

Referring to FIGS. 3, 4 and 6, power for the tip electrode 106 is provided by an insulated power wire 146 that is attached to a portion of the tip electrode base 138 and extends through the catheter lumen 148 to the electrical connector 122 on the handle 108. Cooling fluid is provided to the tip electrode 106, and adjacent tissue, by way of a fluid tube 150 that extends to the handle 108. The distal end of the fluid tube 150 (FIG. 6) is mounted to the insulation member 128 by a connector 152, with a base plate 154, that is preferably formed from a metal such as aluminum or stainless steel or other material of high thermal conductivity for the reasons discussed below. The proximal end of the fluid tube 150 is connected to a valve (not shown) within the handle 108. A fluid inlet tube 156 (FIG. 1) is also connected to the valve, and extends proximally from the handle 108. A connector 158, which may be connected to the fluid supply and control apparatus 300, is mounted on the proximal end of the fluid inlet tube 156. The valve is controlled by a control knob 160 on the handle body 118 which, in turn, allows the clinician to, if necessary, control the fluid flow rate through the valve.

With respect to the temperature sensing performed by the exemplary catheter apparatus 100, first and second temperature sensors 162 and 164 (FIG. 6) may be mounted within the electrode assembly 126 in such a manner that the temperature increase of the fluid passing through the tip electrode 106 may be measured. More specifically, the first temperature sensor 162 is mounted on, and senses the temperature of, the base plate 154 of the connector 152. Because the connector 152 is formed from high thermal conductivity material, is mounted on the insulation member 128, and is separated from the catheter distal portion 112 by air, the temperature of the connector 152 will be equal to temperature of the irrigation fluid as the fluid enters the tip assembly. Thus, by sensing the temperature of the connector 152, the sensor 162 senses the temperature of the irrigation fluid as it enters the tip assembly 126. It should be noted that sensing the temperature of the irrigation fluid at the tip assembly inlet provides more accurate data than, for example, measuring the temperature of the fluid at the fluid supply and control apparatus 300 because the fluid may be heated by body heat as it travels through the catheter 102.

The second temperature sensor 164 is mounted on the inner surface of the tip electrode base portion 138 in the illustrated embodiment. Given the location of the fluid outlets 134 and the high thermal conductivity of the tip electrode 106, the temperature of the electrode base portion 138 will be equal to the temperature of the irrigation fluid when the fluid exits the tip assembly 126. Thus, by sensing the temperature of the electrode base portion 138, the sensor 164 senses the temperature of the irrigation fluid as it exits the tip assembly 126.

In the illustrated embodiment, the temperature sensors 162 and 164 are thermocouples. The thermocouple wires 166 and 168 (FIGS. 3 and 4) from each thermocouple extend through tubes 170 and 172 to the electrical connector 122. It should be noted that the present catheters are not limited any particular temperature sensors. Other suitable temperature sensors include, but are not limited to, thermistors. Also, the tip assembly 126 may be configured such that one or both of the temperature sensors are positioned within the fluid path.

Clearance for the wires that extend to the tip electrode 106 may be provided in a variety of ways. Referring to FIGS. 6 and 7, such clearance is provided in the illustrated embodiment by grooves 174 and 176 that extend along the outer surface of the insulation member 128 and define clearance channels between the inner surface of the catheter distal member 112 and the insulation member.

Turning to the manner in which the present tip assembly 126 may be used to determine how much of the supplied energy is lost to the irrigation fluid, the power supplied to (and dissipated in) the tissue from the electrode 106 (P_{TISSUE}) is equal to the power supplied to the electrode 106 (P_{SUPPLIED}) less the portion of power that is lost to, and heats, the irrigation fluid (P_{LOST}), i.e. P_{TISSUE}=P_{SUPPLIED}−P_{LOST}. The power lost to the irrigation fluid (P_{LOST}) may be determined by measuring the temperature of the fluid as it enters the tip electrode 106 (T_{IN} as sensed by sensor 162) and the temperature of the fluid as it exits the tip electrode (T_{OUT} as sensed by sensor 164). In particular, the power lost to the irrigation fluid, P_{LOST}=ΔT_{FLUID}×Q×ρ×Cp, where ΔT_{FLUID} is T_{OUT}−T_{IN}, Q is the flow rate, ρ is the fluid density, and Cp is the fluid heat capacity. The fluid density and fluid heat capacity of various irrigation fluids may be stored in the ESU controller 220, or may be input by way of the control panel 203, or may be supplied to the ESU directly from the fluid supply apparatus 300. The flow rate may be input into the ESU controller by way of the control panel 203 or may be supplied to the ESU directly from the fluid supply apparatus 300. Once calculated, the magnitude of the actual power being dissipated in the tissue (P_{TISSUE}) may be used by the clinician, and/or the power supply and control apparatus 200, and/or the fluid supply and control apparatus 300 to regulate the procedure.

The exemplary power supply and control apparatus (“power supply”) 200 includes an electrosurgical unit (“ESU”) 202 that supplies and controls RF power. A suitable ESU is the Model 4810A ESU sold by Boston Scientific Corporation of Natick, Mass. The ESU 202 has a power generator 201 and a control panel 203 that allows the user to, for example, set the power level, the duration of power transmission, and a tissue temperature for a given coagulation procedure. The ESU 202 may also be configured to adjust the magnitude of the power being supplied to electrode 106 during an irrigated ablation procedure in such a manner that actual power being dissipated in the tissue (P_{TISSUE}) is equal to the level set by the clinician.

The ESU 202 transmits energy to the electrode 106 by way of a cable 204. The cable 204 includes a connector 206 which may be connected to the catheter electrical connector 122 which, in turn, is connected to the catheter apparatus power and signal wires 124, 146, 166 and 168. The cable 204 also includes a connector 208, which may be connected to a power output port 210 on the ESU 202. Power to the catheter apparatus 100 may be maintained at a constant level during a coagulation procedure, or may be varied, or may substantially reduced or may be shut off completely, depending upon the temperatures measured at the tip electrode 106 by the sensors 162 and 164. It should be noted here that, given the configuration of the tip electrode 106 (and that of electrode 106a), if the flow rate of the irrigation fluid is sufficient to limit the increase in irrigation fluid temperature to 5-10° C., the temperature of the tissue surface may be assumed to be approximately equal to the inlet temperature at the center of the tissue contact portion 136, i.e. the temperature sensed by sensor 162, and the temperature of the tissue surface may be assumed to be approximately equal to the outlet temperature of the irrigation fluid at the base portion 138, i.e. the temperature sensed by sensor 164. The exemplary ESU 202 is capable of performing both unipolar and bipolar tissue coagulation procedures. During unipolar procedures performed with the exemplary system 10 illustrated in FIG. 1, tissue coagulation energy emitted by the electrode 106 is returned to the ESU 202 through an indifferent electrode 212 that is externally attached to the skin of the patient with a patch and a cable 214. The cable 214 includes a connector 216 that may be connected to one of the power return ports 218 on the ESU 202. Preferably, the ESU power output port 210 and corresponding connector 208 have different configurations than the power return port 218 and corresponding connectors 216 in order to prevent improper connections.

The exemplary ESU 202 also includes a controller 220, such as a microprocessor, microcontroller or other control circuitry, that controls the power delivered to the catheter apparatus in accordance with parameters and instructions stored in a programmable memory unit (not shown). Suitable programmable memory units include, but not limited to, FLASH memory, random access memory (“RAM”), dynamic RAM (“DRAM”), or a combination thereof. A data storage unit, such as a hard drive, flash drive, or other non-volatile storage unit, may also be provided. The controller 220 can employ proportional control principles, adaptive control, neural network, or fuzzy logic control principles. In the illustrated implementation, proportional integral derivative (PID) control principles are applied. The controller 220 may be used to perform, for example, conventional temperature and power control functions such as decreasing power when tissue temperature exceeds a set level. The controller 220 may also be used to selectively increase the level of power being supplied to the tip electrode 106 during irrigated ablation procedures, above that set by clinician with control panel 203, in order reduce or eliminate the difference between the power level set by the clinician and supplied to the electrode 106 (P_{SUPPLIED}) and the actual level of power being dissipated in the tissue (P_{TISSUE}). In other words, the power supply 200 may be used to increase the energy supplied to the tip electrode to account for the energy lost to the irrigation fluid (P_{LOST}).

The exemplary fluid supply and control apparatus (“fluid supply”) 300 illustrated in FIG. 1 may be used to supply cooling fluid to the catheter apparatus 100 or other electrophysiology device. The fluid supply 300 includes housing 302, a fluid outlet port 304, a fluid inlet port 306, a reservoir (not shown), and a pump 308 that is connected to the reservoir and the outlet. The fluid outlet port 304 may be coupled to the catheter apparatus connector 158 by a connector tube 310. The fluid inlet port 306 may be connected to a catheter apparatus by a connector tube (not shown) in instances, such as that discussed below with reference to FIGS. 8-13, where the cooling fluid is returned to the fluid supply 300. The pump 308 is capable of different flow rates (e.g. about 1 ml/min to about 30 ml/min). The reservoir may be located within the housing 302, or may be exterior to the housing. The cooling fluid is not limited to any particular type of fluid. In some procedures, the fluid will be an electrically conductive fluid such as saline. A suitable fluid temperature is about 0 to 25° C. and the fluid supply 300 may be provided with a suitable cooling system, if desired, to bring the temperature of the fluid down to the desired level.

The fluid supply 300 also includes a controller 312 that, in the illustrated implementation, receives information such as measured temperature and supplied power from the power supply 200 by way of a connection 314. The connection 314 may be a wired connection, as shown, or may be a wireless connection. The controller 312 in some implementations be configured to adjust the flow rate from the pump 308 based on the difference between the power dissipated in the tissue (P_{TISSUE}) and the power supplied to the electrode 106 (P_{SUPPLIED}) received from the power supply 200. For example, the flow rate of the irrigation fluid may be reduced in order to reduce the amount of power being lost to the cooling fluid (P_{LOST}). The manner in which the controller 312 processes information and derives control signals to control the pump 308 (and flow rate) can vary. For example, the controller 312 can employ proportional control principles, adaptive control, neural network, or fuzzy logic control principles. In the illustrated implementation, proportional integral derivative (PID) control principles are applied.

The principles described above are also applicable to closed irrigated catheters, i.e. catheters in which the irrigation fluid is returned to the proximal end of the catheter instead of being released into the body. One example of such a closed irrigated catheter is generally represented by reference numeral 102a in FIGS. 8 and 9. Catheter 102a is substantially similar to catheter 102 and similar elements are represented by similar reference numerals. The discussion above of such similar elements is incorporated herein by reference.

The exemplary catheter 102a includes a distal member 112 that supports a tip assembly 126a with an electrode 106a and an insulation member 128a that provides thermal and electrical insulation. The tip assembly 126a is configured such that the irrigation fluid which is delivered thereto by way of the fluid tube 150 is returned to the proximal end of the catheter 102a by way of the catheter lumen 148. From there, it is directed through a tube (not shown) similar to the fluid inlet tube 156 in FIG. 1. The tip electrode 106a has a tissue contact portion 136a and a base portion 138a that mounts the tip electrode to the catheter distal member 112. The tissue contact portion 136a is relatively thin to promote heat transfer from the tissue to the fluid within the heating space 132 and is approximately hemispherical-shaped. The base portion 138a is relatively short so that the tip electrode 106 will not be exposed to blood, and the convective cooling effects thereof, during ablation procedures (FIG. 13). The insulation member 128a includes a hemispherical portion 140a and a cylindrical portion 142a, and the inlet lumen 130 extends between both portions. The hemispherical portion 140a is slightly smaller in diameter than the electrode tissue contact portion 136a and, when the two are positioned relative to one another in the manner illustrated in FIG. 9, the fluid heating space 132 is defined therebetween. A plurality of protrusions 178 (FIG. 12) may be provided on the outer surface of the hemispherical portion 140a in order to insure proper spacing between the electrode 106a and insulation member 128a. The outer diameter of the insulation member cylindrical portion 142a is substantially equal to the inner diameters of the catheter distal member 112 and the tip electrode base portion 138a to create a seal therebetween. A plurality of channels 180 (FIGS. 9 and 10) allow fluid to flow from the fluid heating space 132 to the catheter lumen 148.

With respect to temperature sensing, the tip assembly 126a is provided with temperature sensors 162 and 164 that respectively sense the inlet and outlet temperature of the irrigation fluid. To that end, temperature sensor 162 senses the temperature of the connector 152 in the manner described above. With respect to the outlet temperature, the exemplary tip assembly 126a includes a thermally conductive ring 182 (FIGS. 9 and 11) that is carried on the proximal end of the insulation member 128a such that it will be in contact with, and the same temperature as, the fluid flowing past the insulation member to the catheter lumen 148. The temperature sensor 164 senses the temperature of the thermally conductive ring 182. Thermal insulation material 184 separates the connector 152 and temperature sensor 162 from the thermally conductive ring 182 and temperature sensor 164. A tube 186 is provided to protect the thermocouple wires and tubes 170 and 172 from the returning irrigation fluid. Also, in the illustrated embodiment, the thermally conductive ring 182 has a discontinuity (FIG. 11) to accommodate the steering center support 114.

Although the present inventions have been described in terms of the preferred embodiments above, numerous modifications and/or additions to the above-described preferred embodiments would be readily apparent to one skilled in the art. By way of example, but not limitation, the functionality of a power supply and control apparatus 200 and a fluid supply and control apparatus 300 may be incorporated into a single apparatus. It is intended that the scope of the present inventions extend to all such modifications and/or additions and that the scope of the present inventions is limited solely by the claims set forth below.

I claim:

1. An electrophysiology device, comprising:
an elongate body defining a distal region;
an electrode associated with the distal region of the elongate body and defining an inner surface;
a relatively thin fluid heating space associated with the inner surface of the electrode;
an inlet and an outlet associated with the relatively thin fluid heating space; and
first and second temperature sensors respectively in thermal communication with the inlet and the outlet.

2. An electrophysiology device as claimed in claim 1, wherein the elongate body comprises an elongate catheter body.

3. An electrophysiology device as claimed in claim 1, wherein the elongate body defines a distal end and the electrode is mounted on the distal end of the elongate body.

4. An electrophysiology device as claimed in claim 1, wherein the outlet comprises a plurality of outlets.

5. An electrophysiology device as claimed in claim 1, further comprising:
an insulation member defining an outer surface located in spaced relation to the inner surface of the electrode such that the relatively thin fluid heating space is located therebetween.

6. An electrophysiology device as claimed in claim 5, wherein
the inner surface of the electrode is substantially hemispherical in shape; and
the outer surface of the insulation member is substantially hemispherical in shape.

7. An electrophysiology device as claimed in claim 5, wherein the inlet is defined by the insulation member.

8. An electrophysiology device as claimed in claim 1, wherein the electrode defines an outer surface and the outlet extends through the electrode to the outer surface.

9. An electrophysiology device as claimed in claim 1, wherein
the elongate body defines an interior; and
the outlet is associated with the interior of the elongate body.

10. An electrophysiology device as claimed in claim 1, wherein
the electrode defines a diameter that is about 1 mm to 4 mm; and
the relatively thin fluid heating space is about 0.05 to 0.2 thick.

11. An electrophysiology device configured to heat tissue, comprising:
an elongate body defining a distal region;
an irrigated electrode assembly, through which irrigation fluid passes, associated with the distal region of the elongate body and including an electrically and thermally conductive tissue contact surface that receives heat from tissue as it supplies electrical energy to tissue;
means for transferring substantially all of the heat received from the tissue by the tissue contact surface to the irrigation fluid that passes through the electrode assembly;
a first temperature sensor that senses the temperature of the irrigation fluid as it enters the electrode assembly; and
a second temperature sensor that senses the temperature of the irrigation fluid as it exits the electrode assembly.

12. An electrophysiology device as claimed in claim 11, wherein the elongate body comprises an elongate catheter body.

13. An electrophysiology device as claimed in claim 11, wherein the elongate body defines a distal end and the electrode is mounted on the distal end of the elongate body.

14. An electrophysiology device as claimed in claim 11, wherein the irrigated electrode assembly comprises an open irrigated electrode assembly.

15. An electrophysiology device as claimed in claim 11, wherein the irrigated electrode assembly comprises a closed irrigated electrode assembly.

16. A tissue coagulation method, comprising the steps of:
transferring power to tissue with an electrode;
receiving heat from the tissue with the electrode;
cooling the electrode with irrigation fluid;
transferring substantially all of the heat to the irrigation fluid cooling the electrode; and
measuring the change in temperature of the irrigation fluid as it cools the electrode.

17. A method as claimed in claim 16, wherein the step of cooling the electrode comprises cooling the electrode with irrigation fluid in an open irrigation process.

18. A method as claimed in claim 16, wherein the step of cooling the electrode comprises cooling the electrode with irrigation fluid in a closed irrigation process.

19. A method as claimed in claim 16, wherein the step of transferring substantially all of the heat comprises transferring substantially all of the heat to the irrigation fluid cooling the electrode as the irrigation fluid flows through a relatively thin fluid heating space.

20. A method as claimed in claim 16, wherein the step of measuring comprises sensing the temperature of the irrigation fluid as it enters the electrode and as it exits the electrode.
